(19) United States
(12) Patent Application Publication
(10) Pub. No.: US 2006/0095012 A1
(43) Pub. Date: May 4, 2006

(54) Thin protective underwear

(76) Inventor: Richmond R. Cohen, Williamsport, PA (US)

Correspondence Address: AMSTER, ROTHSTEIN & EBENSTEIN LLP, 90 PARK AVENUE, NEW YORK, NY, 10016, US

(21) Appl. No.: 10/978,135

(22) Filed: Oct. 29, 2004

(51) Int. Cl. A61F 13/15

(52) U.S. Cl. 604/367; 604/378

(57) ABSTRACT

Protective underwear including a chassis and an insert. The chassis includes a front waist portion having a first lateral end and a second lateral end, a back waist portion having a first lateral end and a second lateral end, and a crotch portion that longitudinally connects the front waist portion to the back waist portion. The first lateral end of the front waist portion is connected to the first lateral end of the back waist portion and the second lateral end of the front waist portion is connected to the second lateral end of the back waist portion. The insert is attached to the chassis and covers at least the crotch portion of the chassis. The insert has an overall thickness of less than about 4 mm, an absorbent capacity of at least 7.5 g/in² and an acquisition speed of less than about 20 seconds on first and second insults and less than about 30 seconds on a third insult.

### FIELD OF THE INVENTION

The present invention relates to disposable absorbent articles having absorbent inserts, particularly protective underwear having thin absorbent inserts.

### BACKGROUND OF THE INVENTION

Conventional disposable absorbent articles such as baby diapers, adult diapers, and feminine hygiene products typically have multiple layers of absorbent materials or composites. Some of these articles include an insert composed of a topsheet, an absorbent core, and an acquisition/distribution layer (ADL) interposed between the topsheet and absorbent core. The absorbent core is generally a composite of fluff pulp and superabsorbent polymer (SAP) that stores most of the liquid entering the article.

As discreetness is an important issue for wearers of adult absorbent products, diapers that are termed thin are becoming more prevalent. In particular, thin diapers are most desirable in retail adult disposable products, where they will be unobtrusive and comfortable to wear, bringing confidence and dignity to the wearer. A growing product segment for the retail adult user is protective underwear, which may be described as a product resembling underwear that comprises a highly absorbent insert. The challenge in designing such a product is to make the protective underwear look and feel as if it were “regular” underwear while maintaining high absorbency.

Generally, inserts for protective underwear are rendered thin by replacing a significant percentage of the fluff pulp in the absorbent core with SAP and then compressing the absorbent core between two layers of material, such as tissue. Although such techniques are effective in providing a thinner diaper, the absorbent properties of the diaper may be compromised. With the combination of compression and increased SAP content, thin diapers tend to show reduced free swell capacities in addition to slow speeds of fluid acquisition. As a result, such structures are more prone to leakage. Hence, the enhancement in discreetness, comfort and fit provided by using a thin structure may be offset by poor absorbency.

To increase the acquisition rates of thin diapers, enhanced ADL materials may be used. Other types of absorbent structures designed to improve the rate of absorbency while inhibiting wetback or rewet involve multiple layers of fibrous webs having strategically varied average pore sizes. See, for example, U.S. Pat. Nos. 5,728,083, 5,569,226 and 5,505,719.

Others have claimed improved comfort and fit with a diaper with the cross-sectional area of the core, defined as the thickness times the width at the crotch area, not exceeding 0.6 in². See, for example, U.S. Pat. No. 5,098,423. The core also has a minimum swelling capacity. However, a high capacity alone cannot prevent leakage, as the speed of acquiring the fluid is most critical. Furthermore, although the thickness of the core is surely valuable in rendering a product more discreet and comfortable, the width of the core does not contribute to improved discretion or, in some cases, even improved comfort.

Another approach is the use of thin sanitary napkins, such as described in U.S. Pat. No. 6,494,871. The napkin has a thickness of less than 5 mm (about 0.2 inches) and has a penetration time of less than 15 seconds for synthetic menstrual fluid. Although such a product combines the elements of a thin core with rapid fluid acquisition, it is clear to one skilled in the art that the challenge of containing small amounts of menstrual fluid slowly entering a thin pad is less daunting than containing relatively much larger quantities of urine flowing rapidly into a thin pad.

There are other challenges that a thin, discreet insert for an incontinence product such as protective underwear must meet to be effective besides enabling high-speed absorption with a high capacity. Among the other challenges are maintaining low rewet and providing sufficient core integrity. The product must show a low level of rewet or fluid wetback after it has received multiple insults. If this objective were not achieved, the user's skin would not stay dry, raising discomfort and potentially hurting skin health. Furthermore, it is imperative that the thin absorbent core holds together well when wet. With a high percentage of superabsorbent particles in the thin core, which themselves do not impart any integrity to the core, keeping the core intact when wet can be problematic. A core which falls apart or bunches will result in discomfort and failure to accept repeat insults effectively.

Accordingly, there is a need for a discreet, thin protective underwear product for adults that is as thin as possible, while still providing a rapid acquisition speed and a suitable absorbent capacity to receive multiple insults. There is also a need for such a product to be convenient and practical to manufacture.

### SUMMARY OF THE INVENTION

An absorbent article according to an exemplary embodiment of the invention includes a chassis and an insert. The chassis includes a front waist portion having a first lateral end and a second lateral end, a back waist portion having a first lateral end and a second lateral end, and a crotch portion that longitudinally connects the front waist portion to the back waist portion. The first lateral end of the front waist portion is connected to the first lateral end of the back waist portion, and the second lateral end of the front waist portion is connected to the second lateral end of the back waist portion. The insert is attached to the chassis and covers at least the crotch portion of the chassis. The insert has an overall thickness of less than about 4 mm, an absorbent capacity of at least 7.5 g/in² and an acquisition speed of less than about 20 seconds on first and second insults and less than about 30 seconds on a third insult.

In at least one embodiment of the invention, the insert includes a backsheet, a topsheet, an absorbent core disposed between the backsheet and the topsheet, and an acquisition/distribution layer disposed between the absorbent core and the topsheet.

In at least one embodiment of the invention, the absorbent core is made up of about 40% to about 100% superabsorbent polymer (SAP).

In at least one embodiment of the invention, the basis weight of the topsheet is in a range of about 13.5 gsm to about 15 gsm.

In at least one embodiment of the invention, the basis weight of the acquisition/distribution layer is in a range of about 30 gsm to about 60 gsm.

In at least one embodiment of the invention, the thickness of the acquisition/distribution layer is in a range of about 1 mm to about 1.5 mm.

In at least one embodiment of the invention, the basis weight of the absorbent core is in a range of about 250 gsm to about 1000 gsm.

In at least one embodiment of the invention, the thickness of the absorbent core is about 2 mm or less.

In at least one embodiment of the invention, the area of the absorbent core is in a range of about 70 in² to about 130 in².

In at least one embodiment of the invention, the top sheet is made of a nonwoven, an apertured film or a composite of a nonwoven and an apertured film.

In at least one embodiment of the invention, the acquisition/distribution layer comprises a nonwoven or an apertured film.

In at least one embodiment of the invention, the acquisition/distribution layer comprises a three-dimensional apertured film.

In at least one embodiment of the invention, the absorbent core is made of a compressed composite of fluff pulp and superabsorbent polymer.

In at least one embodiment of the invention, the absorbent core is rectangular.

In at least one embodiment of the invention, the absorbent core is hourglass-shaped.

In at least one embodiment of the invention, the backsheet is made of a composite of a fluid impermeable film layer and a nonwoven layer.

In at least one embodiment of the invention, the thickness of the backsheet is 0.2 mm or less.

### BRIEF DESCRIPTION OF THE DRAWINGS

The above and related objects, features and advantages of the present invention will be more fully understood by reference to the following detailed description of the presently preferred, albeit illustrative, embodiments of the present invention when taken in conjunction with the accompanying drawings wherein:

FIG. 1 shows a protective underwear product according to an exemplary embodiment of the invention in a spread-out configuration;

FIG. 2 is a cross-sectional view along the line A-A′ of FIG. 1;

FIG. 3 shows the protective underwear product of FIG. 1 in a fully assembled configuration; and

FIG. 4 shows an absorbent core useable with a protective underwear product according to an exemplary embodiment of the invention.

### DETAILED DESCRIPTION OF PREFERRED EMBODIMENTS

The various exemplary embodiments of the present invention are directed to discreet absorbent articles, such as adult protective underwear products. The protective underwear is as thin as possible, yet contains sufficient absorbent capacity to contain multiple insults of urine, and acquires the urine sufficiently fast so as to prevent leaks. As will be described in further detail below, the thickness of the protective underwear according to various exemplary embodiments of the invention is less that 4 mm, the absorbent capacity is at least 7.5 g/in², and the acquisition time for the first two insults is less than 20 seconds and is less than 30 seconds for the third insult.

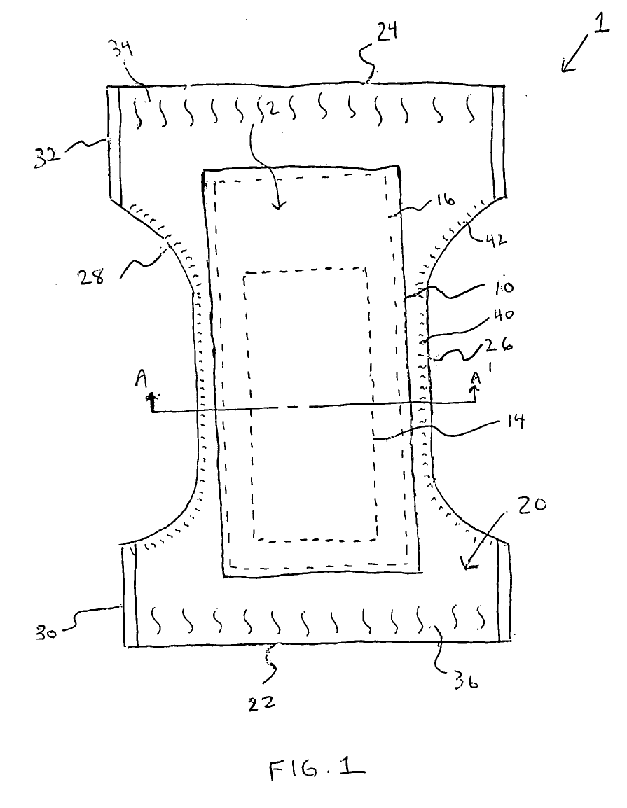

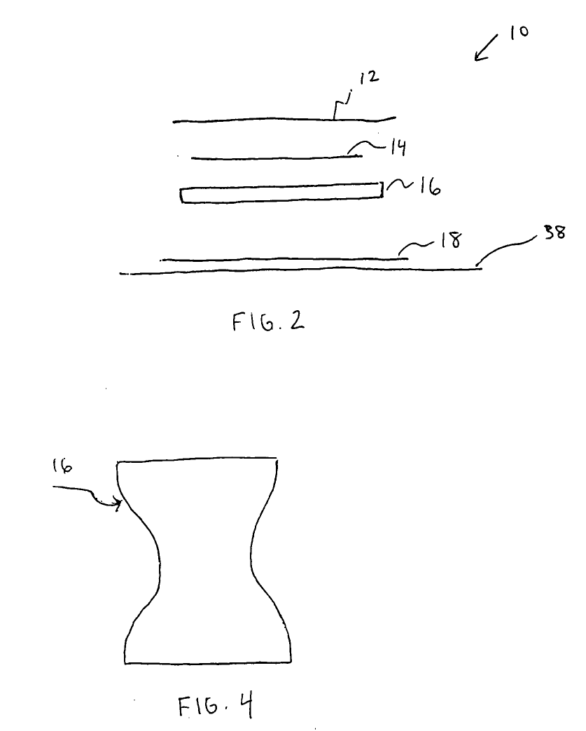

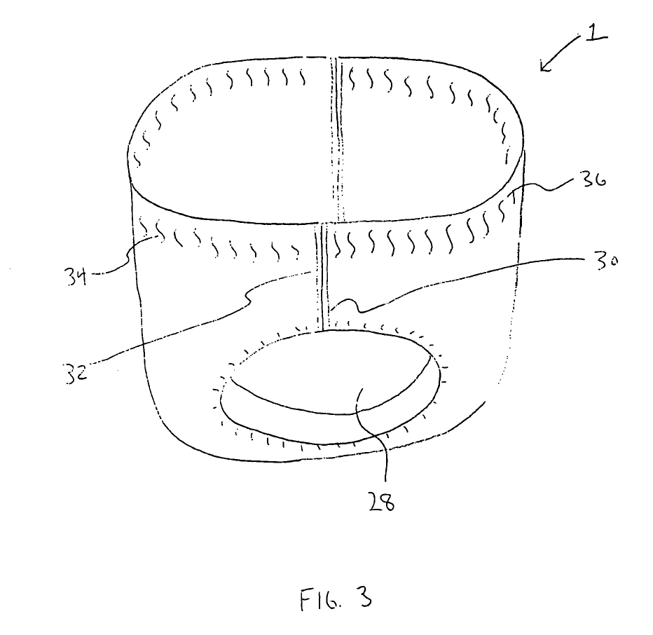

FIGS. 1-3 show the protective underwear according to an exemplary embodiment of the invention. FIG. 1 shows the protective underwear 1 in a spread-out configuration; FIG. 2 shows a cross section of the protective underwear 1 taken along the line A-A′ of FIG. 1; and FIG. 3 shows the protective underwear 1 in a fully assembled configuration.

The protective underwear 1 is composed of an insert 10 and a chassis 20. As shown in FIG. 2, the insert 10 includes a topsheet 12, an ADL 14, an absorbent core 16 and an inner backsheet 18. As described in more detail below, any or all of these components of the insert 10 may contain one or more layers. In addition, a tissue layer can be included between the core and the backsheet. The tissue layer helps with wicking fluid along the length of the absorbent core and prevents pinholes from being formed in the backsheet. The backsheet is particularly vulnerable to having pinholes at the high SAP levels in thin protective underwear.

The chassis 20 includes a front waist portion 22, a back waist portion 24, and a crotch portion 26 that longitudinally connects the front waist portion 22 to the back waist portion 24. As is conventional in such constructions, the crotch portion 26, either alone or in combination with one or both of the back and front waist portions 22, 24, defines leg openings 28 adapted to snugly receive the wearer's legs. The leg openings 28 may be elasticized for a better, more leakproof fit with the wearer's legs. The first lateral ends of the front and back waist portions, 22 and 24, and the second lateral ends of the front and back waist portions are each sealed to form side seals 30 and 32 respectively. With side seals 30 and 32 in combination with leg openings 28 the protective underwear 1 resembles a conventional underwear garment that can be donned simply by putting one's feet through the leg openings and pulling up to the waist. The side seals 30 and 32 are made so as to be easily tearable (releasable), which allows the protective underwear 1 to be quickly removed by performing a simple tearing maneuver. FIG. 1 shows the protective underwear 1 after the side seals have been torn on both sides, with the residual elements of 30 and 32 now displaced. A front elastic element 34 may be formed at the longitudinal edge of the front waist portion 22 and a back elastic element 36 may be formed at the longitudinal edge of the back waist portion 24 to allow the protective underwear 1 to fit snugly around a wearer's waist. In addition, the chassis 20 may include crotch elastics 40 and thigh elastics 42, as shown in FIG. 1.

The chassis 20 contains a nonwoven material that effectively forms an outer backsheet 38. The inner backsheet 18 of the insert 10 is laminated or otherwise bonded to the outer backsheet 38 of the chassis 20. The insert 10 is attached to the chassis 20 so as to extend longitudinally across the crotch portion 26 and partially across the front and back waist portions 22, 24. As shown in FIG. 1, the ADL 14 does not extend the full length of the insert 10 but is strategically positioned closest to the front waist portion 22 so as to enhance the effectiveness of the insert 10 in absorbing body fluids.

The insert 10, and hence the protective underwear 1, preferably have an overall thickness of less than about 4 mm, an absorbent capacity of about 7.5 g/in² and acquisition speeds of less than 20 seconds on the first two insults and less than 30 seconds on the third insult.

The topsheet 12 of the insert 10 is preferably a nonwoven, apertured film or a composite of a nonwoven and an apertured film capable of conducting fluid flow through it yet providing some protection against rewetting of the skin. The apertured film may be a polypropylene or polyethylene based film. The nonwoven is generally comprised of polyolefinic fibers bonded together through a thermal, chemical or hydroentanglement process or formed directly as a spunbond fabric. Preferably, the nonwoven material is comprised of polypropylene fibers that are topically treated with a hydrophilic finish, but such fibers may also contain a hydrophilic internal additive that blooms to the surface over time and provides adequate wettability. Generally, such topsheet materials have a basis weight ranging from about 11 gsm to about 20 gsm, with the preferred range being from about 13.5 to 15 gsm. The thickness of the topsheet within this preferred range is about 0.12 mm to about 0.18 mm.

The ADL 14 may be made of a nonwoven or an apertured film capable of facilitating liquid flow through the insert 10 as well as spreading and distributing fluid throughout the insert 10. The ADL 14 may be a polyester or composite nonwoven material that is thermally bonded or chemically bonded, or it may be a three-dimensional apertured film. The ADL 14 is preferably topically treated with a hydrophilic finish or may contain an internal additive that imparts hydrophilicity to the ADL 14. It is important that the ADL 14 exhibits “durable hydrophilicity”, i.e., the ADL 14 is able to withstand repeat “insults” and be able to maintain hydrophilicity. Thus, it is essential that not all of the wetting agent washes off during the first insult. Further, the ADL 14 preferably has sufficient capacity or loft to allow the relatively thin absorbent core 16 time to absorb the oncoming insult. Hence, the ADL 14 preferably contributes to the bulk of the insert 10 in exchange for the more rapid acquisition speed it enables. In this regard, the weight of the ADL 14 is in the range of about 20 gsm to about 120 gsm, with the preferred range being about 30 gsm to 60 gsm. The thickness of the ADL within the preferred range is about 1 mm to 1.5 mm. As indicated above, the ADL 14 may be made up of more than one layer of material. The ADL 14 performs together with the absorbent core 16 to provide a first and second acquisition time of less than 20 seconds and a third acquisition time of less than 30 seconds.

The absorbent core 16 is generally a compressed composite of fluff pulp and superabsorbent polymer (SAP) but may optionally include synthetic fibers. The absorbent core 16 may also contain no fluff but only SAP with or without synthetic fibers. In general, the SAP preferably makes up at least about 40% to about 100% of the absorbent core 16. Often these materials are sandwiched between two layers of tissue or nonwoven to help contain the SAP. Also, the materials may be held together by hydrogen bonding, thermal bonding, chemical bonding, adhesive lamination or a combination of these mechanisms. The basis weight of the absorbent core 16 is in the range of about 250 gsm to about 1000 gsm. The thickness of the absorbent core 16 is preferably 2 mm or less. Furthermore, the core 16 has an absorbent capacity of at least 7.5 g/in².

The absorbent core 16 may have an hourglass shape, as shown in FIG. 4, with the narrowest width at the crotch, or it may be rectangular, as shown in FIG. 1. The area of the absorbent core 16 may range from about 70 to 130 in² so that the protective underwear can satisfy size requirements for small adults up to bariatric adults. Regardless of the size of the user, the absorbent capacity is at least 7.5 g/in².

The inner backsheet 18 of the protective underwear 1 is preferably comprised of a fluid impermeable film layer and often includes an outer nonwoven layer for improved softness. The film is preferably made of a polyolefin or combination of polyolefins and is generally about 0.02 mm thick. The nonwoven for the inner backsheet 18 is often comparable to the nonwoven for the topsheet 12, with the exception that no hydrophilic additives are needed. The thickness of the nonwoven may range from about 0.12 mm to about 0.18 mm. Hence, the thickness of the backsheet is about 0.2 mm or less.

The protective underwear according to the exemplary embodiments of the invention is thinner than conventional protective underwear, yet is able to exhibit similar and, in some cases, better performance characteristics. This is achieved by the use of a higher percentage of SAP material in the absorbent core, which allows the absorbent core to be compressed into a thinner shape, and by selecting the appropriate ADL system to work synergistically with such a core. As mentioned above, the preferred percentage of SAP material is at least 40%, while conventional protective underwear products include between about 25-50% SAP, but cannot be compressed to be thin for fear of lower acquisition speeds and lower absorbent capacity. In addition, using the preferred materials for the topsheet, ADL and backsheet discussed above further allows the inventive protective underwear product to maintain excellent performance characteristics.

The following test procedures were used in the Examples described below, which illustrate the effectiveness of the protective underwear product according to exemplary embodiments of the present invention:

Thickness

All thickness measurements were made with a Digital Micrometer, Model 49-72, made by TMI (Testing Machines Inc.) of Ronkonkoma, N.Y. The instrument is specially made with a 2″-diameter anvil which applies a load of 95 g/sq. in. to the sample. The thickness is always measured in the crotch area of the protective underwear.

Acquisition Time/Rewet

The test procedure used to evaluate the multiple-insult acquisition time and rewet is similar to others that are widely used in the field. The absorbent structure is laid flat on a surface and elastics are trimmed, if necessary. A dosing ring (60 mm I.D., 70 mm O.D., and 40 mm height) is placed on the targeted area of the absorbent structure. Then, 100 ml of synthetic urine (0.9% NaCl solution) is measured in a graduated cylinder and poured into a 125-ml separatory funnel. The funnel discharges fluid at a rate of 9 ml/s when its stopcock valve is opened fully. Positioning the bottom tip of the funnel 40 mm from the surface of the absorbent structure in the center of the dosing ring, the stopcock is opened, and fluid is dispensed onto the absorbent structure. Simultaneously, a timer is activated. The timer is stopped when the 100-ml dose completely passes through the topsheet. This time is recorded as the first acquisition time.

The dosing ring is now removed and another timer is activated to measure 15 minutes. After 15 minutes, a stack of pre-weighed filter paper (AFI Grade 950, 9-cm diameter) weighing about 10 g is placed in the center of the wetted target area. A cylindrical weight applying 1 psi of pressure is placed on top of the filter paper, with the weight having a diameter also of 9 cm. After waiting 1 minute, the weight is removed, and the filter paper is weighed. The difference in weight is recorded as the first rewet.

Two additional 100-ml doses of synthetic urine are applied using almost the identical procedure as outlined above to produce a total of three “insults” per absorbent structure. For the second and third insults, 15 g of filter paper is used. The total number of replicates is either 5 or 10 per absorbent structure. The average values of the acquisition times and rewets are computed.

Absorbent Capacity/Core Integrity

The test procedure used to measure the absorbent capacity of the absorbent article is also analogous to others used in the field and is often referred to as “Dunk and Drain” or “Dip and Drip”. Enough saline solution as described above is used to fill a shallow tray measuring about 20″×30″ to a depth of about 2″. The protective underwear sample is weighed. The product is immersed in the solution with the topsheet side of the product entering first and is then allowed to soak in the solution for about 10 minutes. The wet product is then placed on a hanging rod across its latitudinal midsection with the topsheet facing up. The product then drains for a period of about 3 minutes. During this drain time, the core may not be able to sustain the weight of the saline absorbed, and under this weight may break apart. The percentage of the core that falls is recorded as the core failure. The product is then placed in a weighing tub, and the wet weight is determined. The absorbent capacity is determined by subtracting the dry weight of the protective underwear from the wet weight, and then by dividing the difference by the area of the absorbent core. For a rectangular core, this area would simply be the length of the core multiplied by the width of the core.

### EXAMPLE 1

A prototype of an insert for a protective underwear product of the invention was assembled. The insert included a 13.5 gsm nonwoven topsheet, product code SB1350021 provided by First Quality Nonwovens of McElhattan, Pa., an apertured polyethylene film ADL with conical pores, known as AquiDry® provided by Tredegar Film Products of Richmond, Va., and a 450 gsm absorbent core known as NovaThin®, with product code J4500755DTNBE, provided by Rayonier Inc. of Jacksonville, Fla., and a liquid impermeable film backsheet. The absorbent core and topsheet were cut to 21″ long and 4.0″ wide. The ADL was cut to 21″ long and 3.25″ wide.

The following were the thickness measurements for the layers of the prototype:
Nonwoven: 0.15 mm
ADL: 1.27 mm
Core: 1.83 mm
Backsheet: 0.03 mm.

The total thickness of the structure is 3.28 mm. Ten samples were measured.

The acquisition times measured were 12.4 s, 13.2 s and 14.6 s for the first, second and third insults, respectively. Five samples were measured to determine the acquisition time. The absorbent capacity was measured to be 9.5 g/in². Ten samples were tested to determine the absorbent capacity.

### EXAMPLE 2

A complete protective underwear product was manufactured to include a prototype insert similar to the prototype insert used in Example 1. The manufactured product included the same grade of nonwoven topsheet and core material as in Example 1. The ADL consisted of a dual-layered apertured film laminate. The layer of apertured film facing the topsheet was the same as that used in Example 1. Laminated to this material was a second apertured film with smaller conical pores, also available from Tredegar Film Products, having the code number 25475. The second apertured film was attached to the core. The backsheet was comprised of an impermeable film provided by Filmtech Corporation of Allentown, Pennsylvania and a 13.5 gsm nonwoven made by First Quality Nonwovens.

The following test results were measured for a sample size of ten in each test:
Thickness: 3.61 mm
First Acquisition Time: 15.9 s
Second Acquisition Time: 17.4 s
Third Acquisition Time: 22.4 s
First Rewet: 0.10 g
Second Rewet: 1.88 g
Third Rewet: 6.26 g
Absorbent Capacity: 7.58 g/in²

### COMPARATIVE EXAMPLE

A conventional protective underwear product was used to obtain rewet and acquisition time test data. The conventional product included an apertured film ADL and an absorbent core made of a mixture of Grade Rayfloc JLD-E fluff pulp provided by Rayonier Inc. and Grade SA55S superabsorbent polymer provided by Sumitomo Seika Chemicals Co. Ltd. of Japan. The absorbent core contained about 25% SAP. The thickness of the conventional product was about 4.93 mm. The data obtained using the conventional protective underwear product is compared to that obtained using the protective underwear product of Example 2 in Table 1 below.

TABLE 1

| | | Rewet (g) | | | Acquisition Time (s) | | | | |
|---|---|---|---|---|---|---|---|---|---|
| | Thickness | 1^{st} | 2^{nd} | 3^{rd} | 1^{st} | 2^{nd} | 3^{rd} | Core | Product |
| | (mm) | Rewet | Rewet | Rewet | Rewet | Rewet | Rewet | Failure (%) | Weight (g) |
| Example 2 | 3.61 | .10 | 1.88 | 6.26 | 15.9 | 17.4 | 22.4 | 0 | 55.9 |
| Comparative Example | 4.93 | .11 | 1.03 | 5.22 | 17.9 | 22.3 | 31.5 | 0 | 66.13 |

As shown in Table 1, the conventional product used in the Comparative Example exhibited longer acquisition times than the protective underwear of Example 2, even though the conventional product was thicker and heavier. Further, although the rewet results for the protective underwear of Example 2 are higher than that of the protective underwear of the Comparative Example, the difference is not statistically significant. It is also important to note here that the comparatively thin structure of the Example 2 product did not have a negative effect on the core failure data. Thus, in general, the data shown in Table 1 demonstrates that the thin protective underwear according to the various exemplary embodiments of the invention is able to exhibit equivalent or even better characteristics than the conventional protective underwear product.

The protective underwear product of the present invention is not limited to the embodiments discussed above, and can include any other structural elements that allow for improved use and fit. For example, in at least one embodiment, the protective underwear can include hook fasteners disposed at the lateral ends of the back waist portion, so that after the side seals are broken, the hook fasteners can be used to refasten the protective underwear around the wearer's waist.

Now that the preferred embodiments of the present invention have been shown and described in detail, various modifications and improvement thereon will become readily apparent to those skilled in the art. Accordingly, the spirit and scope of the present invention is to be construed broadly and limited only by the appended claims, and not by the foregoing specification.

What is claimed is:

1. Protective underwear comprising:
a chassis comprising:
a front waist portion having a first lateral end and a second lateral end;
a back waist portion having a first lateral end and a second lateral end, the first lateral end of the front waist portion being connected to the first lateral end of the back waist portion and the second lateral end of the front waist portion being connected to the second lateral end of the back waist portion; and
a crotch portion that longitudinally connects the front waist portion to the back waist portion; and
an insert attached to the chassis that covers at least the crotch portion of the chassis, the insert having an overall thickness of less than about 4 mm, an absorbent capacity of at least 7.5 g/in² and an acquisition speed of less than about 20 seconds on first and second insults and less than about 30 seconds on a third insult.

2. The protective underwear of claim 1, wherein the insert comprises:
a backsheet;
a topsheet;
an absorbent core disposed between the backsheet and the topsheet; and
an acquisition/distribution layer disposed between the absorbent core and the topsheet.

3. The protective underwear of claim 2, wherein the absorbent core comprises superabsorbent polymer material.

4. The protective underwear of claim 3, wherein the superabsorbent polymer makes up about 40% to about 100% of the absorbent core.

5. The protective underwear of claim 4, wherein the absorbent core further comprises fluff pulp.

6. The protective underwear of claim 4, wherein the absorbent core further comprises synthetic fibers.

7. The protective underwear of claim 4, wherein the thickness of the absorbent core is about 2 mm or less.

8. The protective underwear of claim 4, wherein the basis weight of the absorbent core is in a range of about 250 gsm to about 1000 gsm.

9. The protective underwear of claim 4, wherein the area of the absorbent core is in a range of about 70 in² to about 130 in².

10. The protective underwear of claim 4, wherein the absorbent core is rectangular.

11. The protective underwear of claim 4, wherein the absorbent core is hourglass-shaped.

12. The protective underwear of claim 2, wherein the thickness of the acquisition/distribution layer is in a range of about 1 mm to about 1.5 mm.

13. The protective underwear of claim 2, wherein the basis weight of the acquisition/distribution layer is in a range of about 30 gsm to about 60 gsm.

14. The protective underwear of claim 2, wherein the acquisition/distribution layer is made of a nonwoven or an apertured film.

15. The protective underwear of claim 2, wherein the acquisition/distribution layer is made of a three-dimensional apertured film.

16. The protective underwear of claim 2, wherein the topsheet is made of a nonwoven, an apertured film or a composite of a nonwoven and an apertured film.

17. The protective underwear of claim 2, wherein the basis weight of the topsheet is in a range of about 13.5 gsm to about 15 gsm.

18. The protective underwear of claim 2, wherein the thickness of the backsheet is about 0.2 mm or less.

19. The protective underwear of claim 2, wherein the backsheet is made of a composite of a fluid impermeable film layer and a nonwoven layer.

20. The protective underwear of claim 1, wherein the crotch portion defines leg openings, the leg opening being elasticized.

21. The protective underwear of claim 1, further comprising a front elastic element disposed at a longitudinal end portion of the front waist portion and a back elastic element disposed at a longitudinal end portion of the back waist portion

22. The protective underwear of claim 1, wherein the first lateral ends of the back and front waist portions and the second lateral ends of the front and back waist portions are attached together by releasable side seals.

23. An absorbent insert for protective underwear, comprising:
a backsheet;
a topsheet;
an absorbent core disposed between the backsheet and the topsheet; and
an acquisition/distribution layer disposed between the absorbent core and the topsheet, wherein
the insert has an overall thickness of less than about 4 mm, an absorbent capacity of at least 7.5 g/in² and an acquisition speed of less than about 20 seconds on first and second insults and less than about 30 seconds on a third insult.

24. The protective underwear of claim 23, wherein the absorbent core comprises superabsorbent polymer material.

25. The protective underwear of claim 24, wherein the superabsorbent polymer material makes up about 40% to about 100% of the absorbent core.

26. The protective underwear of claim 25, wherein the absorbent core further comprises fluff pulp.

27. The protective underwear of claim 25, wherein the absorbent core further comprises synthetic fibers.

28. The protective underwear of claim 25, wherein the thickness of the absorbent core is about 2 mm or less.
